(19) United States
(12) Patent Application Publication
(10) Pub. No.: US 2012/0272956 A1
(43) Pub. Date: Nov. 1, 2012

(54) AIRWAY PRESSURE CONTROL DEVICES

(76) Inventor: Michael J. Rusher, Ft. Wayne, IN (US)

(21) Appl. No.: 13/459,564

(22) Filed: Apr. 30, 2012

Related U.S. Application Data
(60) Provisional application No. 61/480,097, filed on Apr. 28, 2011.

(51) Int. Cl. A61M 16/20; A61M 16/10

(52) U.S. Cl. 128/203.12; 128/205.24

(57) ABSTRACT

A positive pressure airway device for providing resistance in an air pathway for a patient exhaling. The device includes an inhalation passageway for letting air into the device when a patient breathing through the device inhales, and an exhalation passageway for passing air out of the device when a patient breathing through the device exhales. The device preferably includes an inhalation valve to allow air to flow into, but not out of, the inhalation passageway, and an exhalation valve to allow air to flow out of the exhalation passageway only when a patient using the device exhales with an expiratory air pressure greater than a pre-determined pressure. A variable pressure spring may control the force needed to open the exhalation valve.

### REFERENCE TO RELATED APPLICATIONS

This application claims the benefit of U.S. Provisional Patent Application Ser. No. 61/480,097, filed Apr. 28, 2011, which is hereby incorporated herein by reference.

### BACKGROUND

Patients that have compromised lungs due to decreased lung capacity resulting from COPD (Chronic Obstructive Pulmonary Disease), CHF (Congestive Heart Failure), or Pulmonary Edema, or decreased lung capacity due to pain or inhibited abdominal diaphragm function, may benefit from therapy such as positive expiratory pressure (PEP) therapy. Patients in need of PEP therapy may not generally exhale with enough force to expand the alveoli. For example, pressures within the alveoli typically range from 4 cmH₂0 to 6 cmH₂0, and when pulmonary capillary pressures (normal range 3 cmH₂0) exceed the alveoli pressures, blood seeps into the alveoli. In this situation it is critical to add pressure greater than 6 cmH₂0 to the space within the alveoli.

Devices that increase expiratory air pressure are known. However, known prior art devices use strictures or small orifices to produce positive expiratory pressures. This may compromise flow with increased friction, requiring more work to exhale. Additionally, some known PEP devices are useful only for allowing a patient to exhale, and may not be used for normal in-and-out breathing.

It is also known that medical ventilators mechanically move breathable air into and out of the lungs, and assist patients who need help breathing or are physically unable to breathe. Such ventilators may pump regular air or oxygen-enriched air to a patient, and are typically connected to a patient's lungs through two tubes through which air may flow: an inspiration tube to provide air/oxygen to the patient's lungs; and an expiration tube to receive exhaled air back from the patient. The inspiration pathway provides air/oxygen that is pumped by the ventilator at a pressure of between 5 and 25 cm of water pressure, depending on the patient's needs. The expiration pathway is passive.

The flow of air (which may be regular, atmospheric air or oxygen-enriched air or some other gas, as desired by medical personnel, all of which will be referred to generically as “air” in this disclosure) is typically controlled by one of two methods. In one method the flow of air is provided under a “pressure control” system in which the flow is provided until it faces a set pressure as detected by a pressure sensor. In the other method the flow of air is provided under a “volume control” system in which the flow is provided until a predetermined volume of air has been delivered. In both cases, the ventilator delivers air at a breath rate (in breaths per minute) that is also set by the ventilator operator.

In some cases a problem may arise if the pressure in the inspiratory tube rises above a level that is safe for the patient. This is particularly a problem when the ventilator is operating in a volume control mode, although excessive pressure may arise even when the ventilator is operating in a pressure control mode.

A need therefore exists for devices that can increase patient safety by providing a positive pressure for expiratory air and/or by preventing the pressure in the inspiratory tube of a medical ventilator from reaching a level that is unsafe for the patient. The present invention addresses those needs.

### SUMMARY OF THE INVENTION

Briefly describing one aspect of the present invention, there is provided a positive pressure airway device for providing resistance in an air pathway for a patient exhaling. In one embodiment the positive pressure airway device comprises or consists essentially of:
a) an inhalation passageway for passing air into the device and to a patient when a patient breathing through the device inhales;
b) an exhalation passageway for passing air from a patient out of the device when a patient breathing through the device exhales;
c) a valve in the inhalation passageway to allow air to flow freely in to a patient when the patient inhales;
d) a valve in the exhalation passageway to allow air to flow out through the device only when a patient using the device exhales with an expiratory air pressure greater than a pre-determined pressure; and
e) an optional mouthpiece for facilitating a patient to inhale and/or exhale through the device.

In some embodiments the valve in the exhalation passageway comprises a stopper to close the passageway and prevent air from flowing through the passageway when the stopper is biased to its closed position, and a stopper-biasing spring to bias the stopper to its closed position unless a pre-determined expiratory air pressure is provided in the passageway. Preferably the force provided against the stopper by the spring is adjustable so that the expiratory air pressure needed to open the exhalation passageway may be varied and selected within the range of about 10 cmH₂0 to about 40 cm/H₂0.

In some embodiments the device may include a spring-retaining housing to retain the stopper-biasing spring and to partially compress the spring to a length shorter than its free length. In certain preferred embodiments the spring-retaining housing is movable with respect to the stopper so that the spring-retaining housing is effective for varying the compression length of the spring, and thus for varying the expiratory air pressure/force needed to open the resistance valve.

The device may be connected to auxiliary air and/or to a nebulizer for providing a drug to the patient when inhaling.

The present invention also provides methods for requiring a patient to breathe out with a pre-determined expiratory air pressure. One such method comprises providing the positive pressure airway device described above, and breathing out through said device with sufficient expiratory air pressure to cause said expiratory air valve to open, allowing air to exit the device and improving patient health.

Another aspect of the invention provides a device for improving the safety of a medical ventilator. The device is positioned in the inspiratory tube that passes air from the ventilator to a patient. The device includes a pressure control valve having an open position and a closed position. Air may not exit the inspiratory tube through the pressure control valve when the valve is in its closed position, but air may exit the inspiratory tube through the pressure control valve when the valve is in its open position. The pressure control valve is biased to its closed position by a pressure spring that may exert a biasing force of between about 5 and about 25 cm of water pressure to maintain the valve in its closed position. The valve may include a diaphragm that is movable to open or close the valve, and the diaphragm may be biased to its closed position by a pressure spring. When air pressure that would be unsafe to the patient builds up in the inspiratory tube, the pressure overcomes the bias of the spring and opens the valve to release the pressure.

The valve may have a first closed position and a second closed position, and may include a safety spring. The pressure spring biases the valve to its first closed position. If the pressure spring fails or is removed, the safety spring biases the valve to its second closed position.

### BRIEF DESCRIPTION OF THE DRAWINGS

FIG. 1 shows a side view, in partial section, of one aspect of the present invention, particularly showing a device for increasing positive pressure within the patient's airways, as the illustrated device is being used to inhale.

FIG. 2 shows a side view, in partial section, of one aspect of the present invention, particularly showing a device for increasing positive pressure within the patient's airways, as the illustrated device is being used to exhale.

FIG. 3 shows an exploded section view of the device of FIGS. 1 and 2.

FIG. 4 shows an end view the device of FIGS. 1 and 2, showing the opening of the inhalation tube and the valve support therein.

FIG. 5 shows a top plan view the device of FIGS. 1 and 2, showing the opening of the exhalation tube and the spring-retaining housing thereon.

FIG. 6 shows a side view the device of FIGS. 1 and 2, with a nebulizer attached to the inhalation opening.

FIG. 7 shows a side view, in partial section, of the device of FIGS. 1 and 2 with the spring-retaining housing being in its compressed position.

FIG. 8 shows a side view, in partial section, of the device of FIGS. 1 and 2 with the spring-retaining housing being in its relaxed position.

FIG. 9 shows an exploded view of the device of FIGS. 1 and 2.

FIG. 10 shows a perspective view of the exhaust/exhalation tube of one aspect of the present invention, showing the threaded outer wall.

FIG. 11 shows a perspective view of the spring-retaining housing of one aspect of the present invention, showing the threaded inner wall.

### DESCRIPTION OF THE PREFERRED EMBODIMENTS

For the purposes of promoting an understanding of the principles of the invention, reference will now be made to certain embodiments and specific language will be used to describe the same. It will nevertheless be understood that no limitation of the scope of the invention is thereby intended, with alterations and modifications being contemplated as would normally occur to persons skilled in the art to which the invention relates.

As indicated above, one aspect of the present invention relates to a device for providing resistance in an air pathway for a patient who is exhaling. In one embodiment the positive pressure airway device comprises:
a) an inhalation passageway for passing air into the device and to a patient when the patient inhales through the device;
b) an exhalation passageway for passing air from a patient out of the device when a patient exhales through the device; and
c) a valve in the exhalation passageway to allow air to flow out through the device only when the patient exhales with an expiratory air pressure greater than 10 cmH₂0. In other embodiments the device includes a valve in the exhalation passageway to allow air to flow out through the device only when the patient exhales with an expiratory air pressure greater than 20 cmH₂0. In still other embodiments the device includes a valve in the exhalation passageway to allow air to flow out through the device only when the patient exhales with an expiratory air pressure greater than 30 cmH₂0.

In some embodiments the valve in the exhalation passageway comprises a stopper to close the passageway and prevent air from flowing through the passageway when the stopper is biased to its closed position, and a stopper-biasing spring to bias the stopper to its closed position unless a pre-determined expiratory air pressure is provided in the passageway. Preferably the force provided against the stopper by the spring is adjustable so that the expiratory air pressure needed to open the exhalation passageway may be varied and selected within the range of 10 cmH₂0 to 30 cm/H₂0.

In a further embodiment the device includes a spring-retaining housing to retain a stopper-biasing compression coil spring and to partially compress the spring to a length shorter than its free length. In certain preferred embodiments the spring-retaining housing is movable with respect to the stopper so that the spring-retaining housing is effective for varying the compression length of the spring, and thus for varying the expiratory air pressure/force needed to open the resistance valve.

In one preferred embodiment the device comprises or consists essentially of:
a) a central tube region;
b) a first passageway for passing air into said central tube region when a patient breathing through the device inhales;
c) a second passageway for passing air out of said central tube region when a patient breathing through the device exhales;
d) a third passageway for passing air from said central tube region and into a patient when the patient breathing through the device inhales, and for passing air from said patient to said central tube when the patient breathing through the device exhales;
e) a one-way resistance valve in said first passageway to allow air to flow in through the first passageway to the central tube when a patient using the device inhales, and to prevent air from flowing out through the first passageway when a patient using the device exhales;
f) a one-way resistance valve in said second passageway to allow air to flow out from the second passageway when a patient using the device exhales with an expiratory air pressure greater than a pre-determined pressure, and to prevent air from flowing in through the second passageway when a patient using the device inhales.

In some embodiments the one-way resistance valve in said second passageway comprises or consists essentially of a stopper to close the passageway and prevent air from flowing through the passageway when the stopper is biased to its closed position, and a stopper-biasing spring to bias the stopper to its closed position unless a pre-determined expiratory air pressure is provided in the passageway.

In some embodiments the stopper-biasing spring is a compression coil spring.

In some embodiments the device comprises or consists essentially of the devices described above, but including a spring housing to retain said stopper-biasing compression coil spring and to partially compress the spring to a length shorter than its free length. The spring housing may be movable with respect to the stopper so that the spring housing is effective for varying the compression length of the spring.

In a further embodiment the device includes a nebulizer for providing a drug to said first passageway.

In another embodiment of the present invention there is provided a method for requiring a patient to breathe out with a pre-determined expiratory air pressure. The method preferably comprises:
a) providing a device for providing resistance in an air pathway for a patient exhaling, the device comprising:
   i) a central tube region;
   ii) a first passageway for passing air into said central tube region when a patient breathing through the device inhales;
   iii) a second passageway for passing air out of said central tube region when a patient breathing through the device exhales;
   iv) a third passageway for passing air from said central tube region and into a patient when the patient breathing through the device inhales, and for passing air from said patient to said central tube when the patient breathing through the device exhales;
   v) an inspiratory air valve in said first passageway to allow air to flow in through the first passageway to the central tube when a patient using the device inhales, and to prevent air from flowing out through the first passageway when a patient using the device exhales; and
   vi) an expiratory air valve in said second passageway to allow air to flow out from the second passageway when a patient using the device exhales with an expiratory air pressure greater than a pre-determined pressure, and to prevent air from flowing in through the second passageway when a patient using the device inhales;
b) breathing out through said device with sufficient expiratory air pressure to cause said expiratory air valve to open, allowing air to exit the device.

In another embodiment the method uses a device that includes an expiratory air valve that includes:
i) a stopper to close the passageway and prevent air from flowing through the passageway when the stopper is biased to its closed position, and
ii a stopper-biasing spring to bias the stopper to its closed position unless a pre-determined expiratory air pressure is provided in the passageway;
and wherein said breathing step includes breathing out with an expiratory air pressure that is at least as great as the pre-determined expiratory air pressure.

In another embodiment the method uses a device that includes a spring housing to retain said stopper-biasing compression coil spring and to partially compress the spring to a length shorter than its free length; wherein said spring housing is movable with respect to the stopper so that the spring housing is effective for varying the compression length of the spring; and wherein said method includes the step of selecting a pre-determined expiratory air pressure and moving the spring housing with respect to the stopper so that the pressure necessary to move the topper to its open position is the pre-determined expiratory air pressure.

In some embodiments of the invention the method requires a pre-determined expiratory air pressure of between 10 cmH₂0 and 30 cm/H₂0. For example, some embodiments use a valve in the exhalation passageway that allows air to flow out through the device only when the patient exhales with an expiratory air pressure greater than 10 cmH₂0. In other embodiments the device uses a valve in the exhalation passageway that allows air to flow out through the device only when the patient exhales with an expiratory air pressure greater than 20 cmH₂0. In still other embodiments the device uses a valve in the exhalation passageway that allows air to flow out through the device only when the patient exhales with an expiratory air pressure greater than 30 cmH₂0.

It is to be appreciated that the present invention provides a device that is designed to increase positive pressure within the patient's airways during exhalation. This expands the lungs within patients that have compromised lungs due to decreased lung capacity resulting from COPD (Chronic Obstructive Pulmonary Disease), CHF (Congestive Heart Failure), Pulmonary Edema, or decreased lung capacity due to pain or inhibited abdominal diaphragm function. The use of the inventive positive pressure airway device (PPAD, optionally referred to as a pneumatic positive expiratory pressure device, or PPEPD) still requires physical effort from the patient, but decreases the amount of physical effort to achieve the desired alveoli expansion. This provides a therapy designed to decrease danger to the patient due to and during cardiopulmonary compromise listed above, and to prevent pulmonary complications due to compromised lung function.

Patients can do this with the positive pressure airway device in any situation. By increasing the pressure provided by the PPAD above 6 cmH20 in the alveoli, this pushes the blood from the Alveoli back into the pulmonary capillaries. Higher pressures will achieve this in a faster manner. The PPAD is designed to function between 10 cmH₂0 and 30 cmH₂0.

In some embodiments of the invention the device uses a valve in the exhalation passageway that prevents the patient from exhaling through the device unless the expiratory air pressure is at least 10 cmH₂0. In other embodiments the device includes a valve in the exhalation passageway to allow air to flow out through the device only when the patient exhales with an expiratory air pressure greater than 15 cmH₂0. In other embodiments the device includes a valve in the exhalation passageway to allow air to flow out through the device only when the patient exhales with an expiratory air pressure greater than 20 cmH₂0. In other embodiments the device includes a valve in the exhalation passageway to allow air to flow out through the device only when the patient exhales with an expiratory air pressure greater than 25 cmH₂0. In still other embodiments the device includes a valve in the exhalation passageway to allow air to flow out through the device only when the patient exhales with an expiratory air pressure greater than 30 cmH₂0. In yet other embodiments the device includes a valve in the exhalation passageway to allow air to flow out through the device only when the patient exhales with an expiratory air pressure greater than 35 cmH₂0. In still other embodiments the device includes a valve in the exhalation passageway to allow air to flow out through the device only when the patient exhales with an expiratory air pressure greater than 40 cmH₂0. More preferably, the device includes a valve that is variable with respect to the necessary expiratory air pressure so that the necessary expiratory air pressure may be selected to be essentially anywhere within the range of 10 cmH₂0 to 40 cmH₂0, or most preferably within the range of 10 cmH₂0 to 35 cmH₂0

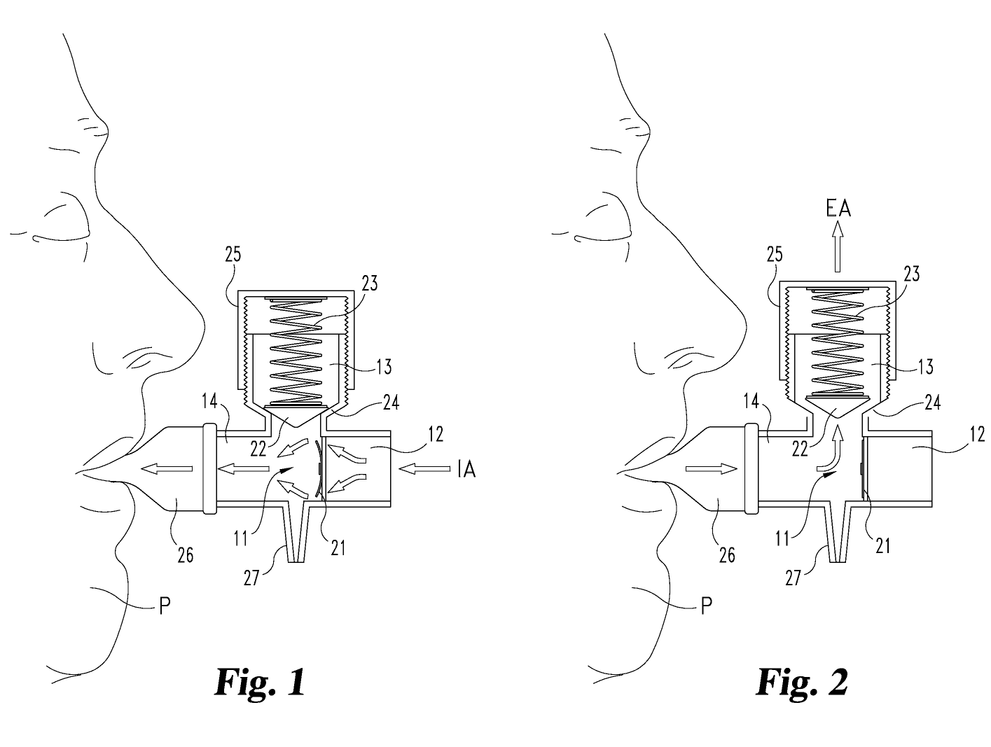

Referring now to the drawings, FIGS. 1-2 show a side view of one embodiment of a positive pressure airway device, in partial section. The illustrated device includes a central tube portion 11 where a first passageway 12, a second passageway 13, and a third passageway 14 meet. First passageway 12 is the “inhalation” passageway through which air may enter the device when a patient using the device inhales. First passageway 12 may include an inhalation valve 21 that allows air to flow in through first passageway 12 to central tube portion 11 when a patient using the device inhales. Valve 21 may also prevent air from flowing out through first passageway 12 when a patient using the device exhales.

Second passageway 13 is the “exhalation” passageway through which air may leave the device when a patient using the device exhales. Second passageway 13 may include a variable-pressure exhalation valve 22 that allows air to flow out through second passageway 13 when a patient using the device exhales. Valve 22 may also prevent air from flowing in through second passageway 13 when a patient using the device inhales.

Third passageway 14 is the “patient breathing” passageway through which air passes into and out of the patient's lungs. Third passageway 14 receives air from first passageway 11 through central tube portion 11 when the patient inhales, and passes air out to second passageway 13 through central tube portion 11 when the patient exhales.

One or more valves may be used to control the air flow. As previously indicated, valve 21 may be used to allow air to flow in through first passageway 12 to central tube portion 11 when a patient using the device inhales. Valve 21 may also prevent air from flowing out through first passageway 12 when a patient using the device exhales. Similarly, valve 22 may allow air to flow out through second passageway 13 when a patient using the device exhales. Valve 22 may also prevent air from flowing in through second passageway 13 when a patient using the device inhales.

Valve 22 is preferably variable with respect to the pressure needed to open the valve. Most preferably valve 22 is biased closed with a pressure of between 10 cmH₂0 and 30 cm/H₂0. The pressure needed to open the valve is selectable, so that when the patient selects an opening pressure of 10 cmH₂0 to open the valve the valve will open when the patient exhales with an expiratory air pressure of at least 10 cmH₂0. Similarly, when the patient selects an opening pressure of 30 cmH₂0 to open the valve the valve will open when the patient exhales with an expiratory air pressure of at least 30 cmH₂0.

Accordingly, it can be seen in FIG. 1 that valve 21 opens when the patient inhales through the device, and it an be seen in FIG. 2 that valve 21 closes on exhalation. Similarly, it can be seen in FIG. 1 that valve 22 remains biased closed when the patient inhales through the device, and it an be seen in FIG. 2 that valve 22 opens when the expiratory air pressure exceeds the selected spring pressure. This combination of valves forces the patient's air to exit through the expiratory pressure exhaust port by forcing the expiratory pressure valve to push open against the pressure control spring.

To further illustrate the operation of valve 22, the valve may comprise a stopper 22 that seats in a lower, sloped portion of sidewall 24 in passageway 13. Spring 23 biases stopper 22 downward with a pressure equal to the expiratory air pressure that is desired.

The pressure exerted by spring 23 may be variable. For example, a spring-retaining housing 25 may be used to vary the compression applied to spring 23, and thereby to vary the pressure needed to move stopper 22 to its open position. Threaded outer sidewalls on exhalation tube 24 may cooperate with threaded inner sidewalls of spring-retaining housing 25 to vary the length of passageway 13, and thus the pressure exerted by spring 23.

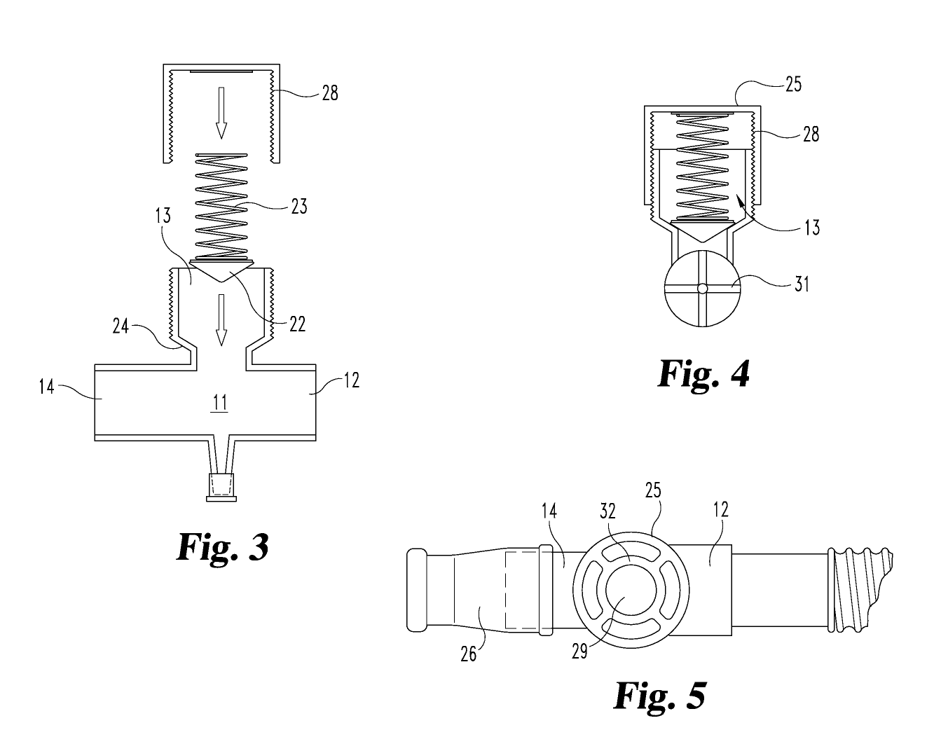

FIG. 3 shows an exploded section view of the device of FIGS. 1 and 2. Spring 23 is positioned above stopper 22 and presses down on stopper 22 when spring-retaining housing 25 is screwed onto tube 24.

FIG. 4 shows an end view the device of FIGS. 1 and 2, showing the opening of the inhalation tube and the valve support 31 therein.

FIG. 5 shows a top plan view the device of FIGS. 1 and 2, showing the opening of the exhalation tube and the spring-retaining housing 25 thereon. Spring-retaining housing 25 includes openings 29 to allow expiratory air to exit the device, and retaining arms 32 to retain the spring in the housing.

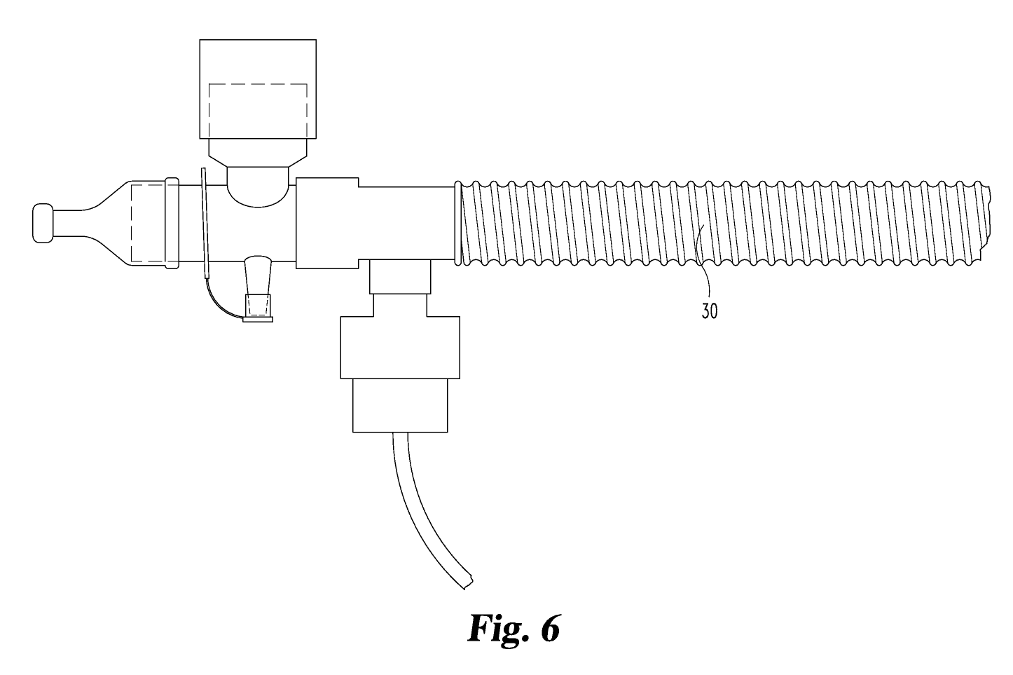

FIG. 6 shows a side view the device of FIGS. 1 and 2, with a nebulizer 30 attached to the inhalation opening.

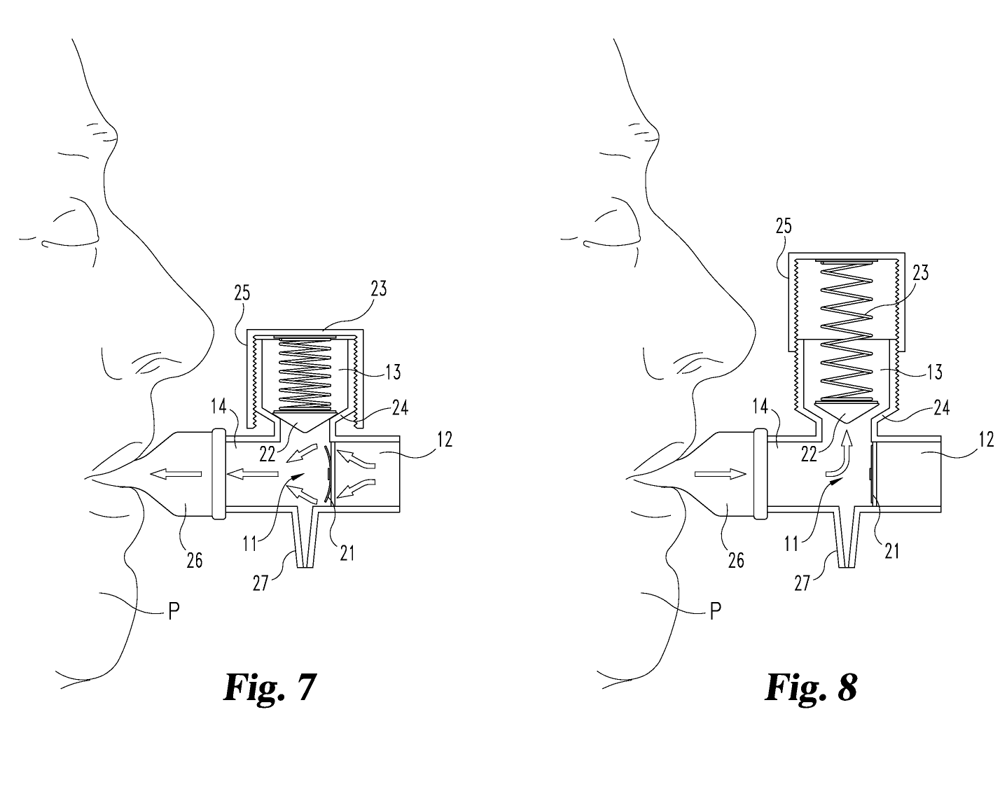

FIG. 7 shows a side view, in partial section, of the device of FIGS. 1 and 2 with the spring-retaining housing being in its compressed position. In the illustrated condition the patient is inhaling and air is entering the device as stopper 22 remains seated to seal exhalation tube 24 closed.

FIG. 8 shows a side view, in partial section, of the device of FIGS. 1 and 2 with the spring-retaining housing being in its relaxed position. In the illustrated condition the patient is exhaling and air is leaving the device as stopper 22 is pushed upward by an expiratory air pressure that exceeds the downward pressure provided by spring 23.

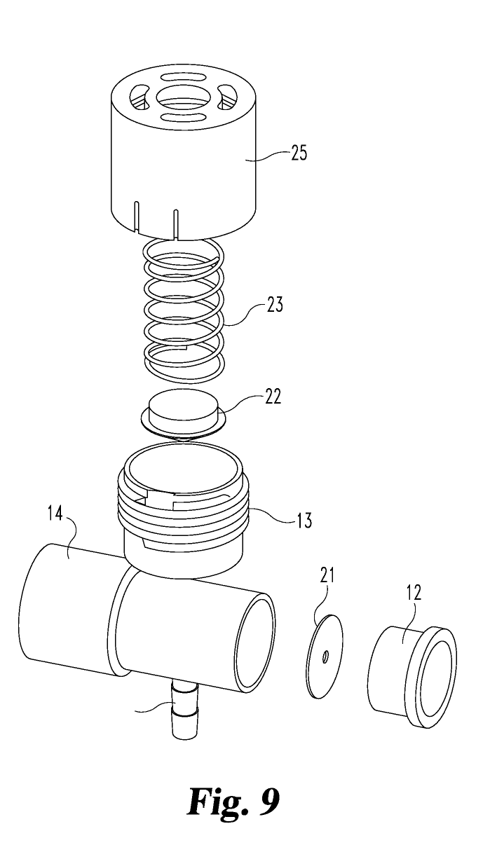

FIG. 9 shows an exploded view of the device of FIGS. 1 and 2.

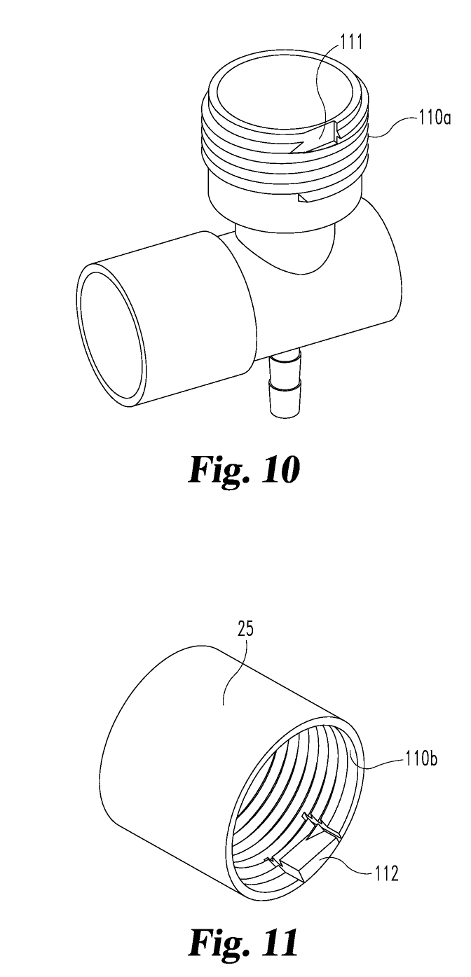

FIG. 10 shows a perspective view of the exhaust/exhalation tube of one aspect of the present invention, showing the threaded outer wall. Threads 110 may include a cut-out 111 to receive a ramp 112. Ramp 112 and cut-out 111 comprise a ramp-lock to lock housing 25 onto tube 24 and prevent the housing from being removed from the tube unless the ramp-lock is released.

FIG. 11 shows a perspective view of the spring-retaining housing of one aspect of the present invention, showing the threaded inner wall. A ramp 112 may be included to lock the housing 25 onto tube 24 unless the user releases the ramp-lock assembly.

As shown in several Figures, an O₂ nipple adapter 27 may be used to facilitate the supply of supplemental oxygen (or other gas) to the patient if and when needed. The nipple adaptor allows supplemental gas to be provided to the patient at any range from less than 1 liter per minute to at least about 15 liters per minute.

It can be seen from the foregoing Figures that valve 21 may include a diaphragm that is deflected inward to allow air to enter during inhalation. When exhaling, that diaphragm presses against support 31 to prevent air from exiting through that opening. Instead, air is forced to exit through the exhalation control valve which provides a positive airway pressure against the patient. When the patient blows with sufficient force, the biasing force of the pressure control spring is overcome and air may exit through the exhalation ports. The positive airway pressure may be controlled within limits by using the pressure control knob to shorten or lengthen the space in the upper housing, thus increasing or decreasing the pressure provided by the spring.

It can be seen from the above that the present invention allows the patient to both inhale and exhale through the device. The device may therefore be used as for normal breathing, without manipulating the device in any way and without requiring the patient to put the device aside to inhale.

It can also be seen from the above that various benefits may be provided by one or more of the various disclosed embodiments. For example, a patient may achieve positive pressure exhalation without compromising expiratory air flow. This provides the benefit of requiring less work by the patient for breathing by (APPE) active positive pressure exhalation. Exhalation is normally passive.

It is known to the art that about two-thirds to three-quarters of a patient's breathing time is spent in exhalation with normal lung function. The inventive PPAD uses exhalation to advantage with positive pressure exhalation. This also creates a normal I/E ratio when the patient is in distress preventing hyperventilation.

The PPAD may be used for expiratory positive pressure ventilation (EPPV) or positive exhalation pressure (PEP). The device is designed to relieve difficulty of breathing at onset of respiratory distress by means of APPE or FPPE (forced positive pressure exhalation) with asthma attacks. This is comparable to the function of PEP with a broader explanation of uses of EPPV or PEP.

The PPAD may also be used for simple lung expansion exercises for patients who have compromised lung function due to restriction and or pain from thoracic and abdominal surgeries.

The PPAD may be used for early intervention of patients who are pending respiratory distress. These patients can benefit greatly from EPPV to prevent or recover from respiratory distress in a short period of time.

The PPAD may prevent air trapping by splinting the bronchiole tubes during APPE.

The PPAD may allow for better ventilation and oxygenation.

The PPAD may act as an internal splint in the smaller bronchiole walls and alveoli to prevent respiratory distress with pulmonary edema resulting from CHF causing tremendous negative pressures within the airways. Respiratory distress may be minimized by recruiting and hyper inflating alveoli during APPE.

The PPAD may help patients expand hypo inflated lungs due to lack of proper deep breathing.

The PPAD may help hold the normal shape of alveoli during exhalation with patients who suffer from obstructive lung disease by splinting the flaccid air sacs and damaged bronchiole tubes. The result may be less stagnant lungs which will help mobilize secretions (increased expansion and contraction of the lungs).

The PPAD may achieve desired pressure without compromising flow. The result may be less energy expended during device use resulting in greater chances of recovery.

The PPAD may be used with supplemental oxygen or an aerosol nebulizer if desired by patient or medical personnel.

In another embodiment of the present invention there is provided a device for improving the safety of a medical ventilator. The device is preferably positioned in the inspiratory tube that passes air from the ventilator to a patient. Alternatively, the device may be positioned in the ventilator itself, such as in an outlet or connector where the ventilator connects to the inspiratory tube. In any case, the device is positioned in the pathway through which air is pumped to the patient's lungs, between the ventilator pump and the patient.

The device includes a pressure control valve having an open position and a closed position. The valve is adapted to ensure that air may not exit the inspiratory tube through the pressure control valve when the valve is in its closed position. This allows the normal functioning of the ventilator and the inspiration tube when the air pressure in the tube is safe for the patient. The valve is also adapted to ensure that air may exit the inspiratory tube through the pressure control valve when the valve is in its open position. The valve is designed to ensure that the valve is in its open position when the air pressure in the inspiration tube is too high for patient safety.

The pressure control valve is biased to its closed position by a pressure spring that exerts a biasing force of between about 5 and about 25 cm of water pressure to maintain the valve in its closed position. The valve may include a diaphragm that is movable to open or close the valve, and the diaphragm may be biased to its closed position by a pressure spring. When air pressure that would be unsafe to the patient builds up in the inspiratory tube, the pressure overcomes the bias of the spring and opens the valve to release the pressure.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, it is to be appreciated that the present invention may comprise or consist essentially of any or all of the illustrated or described devices. For example, the present invention includes devices comprising each of the embodiments illustrated in FIGS. 1 through 11, and the present invention includes devices consisting essentially of any of the embodiments illustrated in FIGS. 1 through 11.

1. A device for providing resistance in an air pathway for a patient exhaling, the device comprising:
a) a central tube region;
b) a first passageway for passing air into said central tube region when a patient breathing through the device inhales;
c) a second passageway for passing air out of said central tube region when a patient breathing through the device exhales;
d) a third passageway for passing air from said central tube region and into a patient when the patient breathing through the device inhales, and for passing air from said patient to said central tube when the patient breathing through the device exhales;
e) a valve in said first passageway that allows air to flow in through the first passageway to the central tube when a patient using the device inhales, and that prevents air from flowing out through the first passageway when a patient using the device exhales;
f) a valve in said second passageway that allows air to flow out from the second passageway when a patient using the device exhales with an expiratory air pressure greater than a selected pressure, that prevents air from flowing out through the second passageway when a patient exhales with an expiratory air pressure that is less than a selected air pressure, and that prevents air from flowing in through the second passageway when a patient using the device inhales.

2. A device according to claim 1 wherein said first passageway, said second passageway, and said third passageway are separate and distinct from each other.

3. A device according to claim 1 wherein said valve in said second passageway includes:
i) a stopper to close the passageway and prevent air from flowing through the passageway when the stopper is biased to its closed position, and
ii a stopper-biasing spring to bias the stopper to its closed position unless the expiratory air pressure in the passageway is greater than a selected pressure;

4. A device according to claim 3 wherein said device includes a spring housing to retain said stopper-biasing spring and to partially compress the spring to a length shorter than its free length.

5. A device according to claim 1 wherein said spring housing is movable with respect to the stopper so that movement of the spring housing is effective for varying the compression length of the spring, and thus is effective for varying the expiratory air pressure that will cause the valve to open.

6. A device according to claim 5 wherein said spring housing is movable with respect to the stopper so that movement of the spring housing is effective for varying expiratory air pressure within the range of 5 psi to 25 psi.

7. A device according to claim 1 and further including a nebulizer for providing a drug to said first passageway.

8. A method for requiring a patient to breathe out with a pre-determined expiratory air pressure, said method comprising:
a) providing a device for providing resistance in an air pathway for a patient exhaling, the device comprising:
i) a central tube region;
ii) a first passageway for passing air into said central tube region when a patient breathing through the device inhales;
iii) a second passageway for passing air out of said central tube region when a patient breathing through the device exhales;
iv) a third passageway for passing air from said central tube region and into a patient when the patient breathing through the device inhales, and for passing air from said patient to said central tube when the patient breathing through the device exhales;
v) an inspiratory air valve in said first passageway to allow air to flow in through the first passageway to the central tube when a patient using the device inhales, and to prevent air from flowing out through the first passageway when a patient using the device exhales; and
vi) an expiratory air valve in said second passageway to allow air to flow out from the second passageway when a patient using the device exhales with an expiratory air pressure greater than a pre-determined pressure, and to prevent air from flowing in through the second passageway when a patient using the device inhales;
b) breathing out through said device with sufficient expiratory air pressure to cause said expiratory air valve to open, allowing air to exit the device.

9. A method according to claim 7 wherein said device includes an expiratory air valve that includes:
i) a stopper to close the passageway and prevent air from flowing through the passageway when the stopper is biased to its closed position, and
ii a stopper-biasing spring to bias the stopper to its closed position unless a pre-determined expiratory air pressure is provided in the passageway;
and wherein said breathing step includes breathing out with an expiratory air pressure that is at least as great as the pre-determined expiratory air pressure.

10. The method of claim 8 wherein said device includes a spring housing to retain said stopper-biasing compression coil spring and to partially compress the spring to a length shorter than its free length; wherein said spring housing is movable with respect to the stopper so that the spring housing is effective for varying the compression length of the spring; and wherein said method includes the step of selecting a pre-determined expiratory air pressure and moving the spring housing with respect to the stopper so that the pressure necessary to move the stopper to its open position is the pre-determined expiratory air pressure.

11. The method of claim 9 wherein the pre-determined expiratory air pressure is at least 25 psi.

12. A positive pressure airway device, consisting essentially of:
a) an inhalation passageway for passing air into the device and to a patient when a patient breathing through the device inhales;
b) an exhalation passageway for passing air from a patient out of the device when a patient breathing through the device exhales;
c) a valve in the exhalation passageway to allow air to flow out through the passageway only when a patient using the device exhales with an expiratory air pressure greater than 10 cmH₂0; and
d) a mouthpiece to facilitate a patient's inhaling and exhaling through said inhalation passageway and said exhalation passageway.

13. A device according to claim 12 wherein said valve in the exhalation passageway includes:
i) a stopper to close the passageway and prevent air from flowing through the passageway when the stopper is biased to its closed position, and
ii a stopper-biasing spring to bias the stopper to its closed position unless the expiratory air pressure in the passageway is greater than a selected pressure;
and wherein said device includes a spring housing to retain said stopper-biasing spring and to partially compress the spring to a length shorter than its free length;
and wherein said spring housing is movable with respect to the stopper so that movement of the spring housing is effective for varying the compression length of the spring, and thus is effective for varying the expiratory air pressure that will cause the valve to open;
and wherein said spring housing is movable with respect to the stopper so that movement of the spring housing is effective for varying expiratory air pressure within the range of 5 psi to 25 psi.

14. A positive pressure airway device, consisting essentially of:
a) a passageway for passing air into the device and to a patient when a patient breathing through the device inhales, and for passing air from a patient out of the device when a patient breathing through the device exhales, said passageway terminating at one end in a mouthpiece;
b) a valve in said passageway to allow air to flow freely into said passageway when a patient inhales, but to allow air to flow out through the passageway only when a patient using the device exhales with an expiratory air pressure greater than 10 cmH₂0, wherein said valve comprises:
i) a stopper to close the passageway and prevent air from flowing through the passageway when the stopper is biased to its closed position, and
ii a stopper-biasing spring to bias the stopper to its closed position unless the expiratory air pressure in the passageway is greater than a selected pressure;
and wherein said device includes a spring retaining housing to retain said stopper-biasing spring and to partially compress the spring to a length shorter than its free length;
and wherein said spring retaining housing is movable with respect to the stopper so that movement of the spring housing is effective for varying the compression length of the spring, and thus is effective for varying the expiratory air pressure that will cause the valve to open;
and wherein said spring housing is movable with respect to the stopper so that movement of the spring housing is effective for varying expiratory air pressure within the range of 5 psi to 25 psi.

15. A device for improving the safety of a medical ventilator, comprising an inspiratory tube for passing air from a ventilator to a patient, said inspiratory tube having a ventilator end and a patient end, and a pressure control valve positioned in said inspiratory tube, said valve having an open position and a closed position,
wherein air may not exit the inspiratory tube through the pressure control valve when the valve is in its closed position, but air may exit the inspiratory tube through the pressure control valve when the valve is in its open position,
wherein said pressure control valve is biased to its closed position by a pressure spring that exerts a biasing force of between about 5 psi and about 25 psi to maintain the valve in its closed position unless the air pressure in the inspiratory tube exceeds the biasing force; and
wherein said pressure control valve moves to its open position when the air pressure in the inspiratory tube exceeds the biasing force, thus allowing air to exit the inspiratory tube through the pressure control valve.

16. The device of claim 15 wherein said valve includes a diaphragm that is movable to open or close the valve.

17. The device of claim 16 wherein said pressure control spring exerts a biasing force on said diaphragm to maintain the valve in its closed position unless the air pressure in the inspiratory tube exceeds the biasing force.

18. The device of claim 15 wherein said pressure control valve has a first closed position and a second closed position differing from said first closed position, wherein said pressure spring biases the valve to its first closed position, and wherein the device further includes a safety spring positioned to push the valve to its second closed position with a force that is less than the biasing force of the pressure spring.

19. A method for improving the safety of a medical ventilator, comprising the steps of:
a) providing a medical ventilator having an inspiratory tube for passing air from the ventilator to a patient, said inspiratory tube having a ventilator end and a patient end;
b) providing a pressure control valve in said inspiratory tube, said valve having an open position and a closed position,
wherein said valve is constructed such that air may not exit the inspiratory tube through the pressure control valve when the valve is in its closed position, but air may exit the inspiratory tube through the pressure control valve when the valve is in its open position, wherein said pressure control valve is biased to its closed position by a pressure spring that exerts a biasing force of between about 10 cm H2O and about 30 cm H₂O to maintain the valve in its closed position unless the air pressure in the inspiratory tube exceeds the biasing force; and
wherein said pressure control valve moves to its open position when the air pressure in the inspiratory tube exceeds the biasing force, thus allowing air to exit the inspiratory tube through the pressure control valve;
c) passing air through said inspiratory tube at a pressure that is less than the biasing force of the pressure spring, thus passing all of that air through the inspiratory tube without exiting through the pressure control valve;
d) allowing some air to flow into said inspiratory tube at a pressure that is greater than the biasing force of the pressure spring, thus causing the pressure control valve to open and allowing air to exit the inspiratory tube through the pressure control valve.
